(19) United States
(12) Patent Application Publication
(10) Pub. No.: US 2021/0386827 A1
(43) Pub. Date: Dec. 16, 2021

(54) CELL-FREE COMPOSITIONS AND METHODS FOR RESTORATION OR ENHANCEMENT OF TISSUE FUNCTION

(71) Applicant: Avery Therapeutics, Inc., Tucson, AZ (US)

(72) Inventors: Jennifer Watson Koevary, Tucson, AZ (US); Jordan J. Lancaster, Tucson, AZ (US); Steven Goldman, Tucson, AZ (US)

(21) Appl. No.: 17/285,853

(22) PCT Filed: Oct. 15, 2019
(86) PCT No.: PCT/US19/56390

§ 371 Date: Apr. 15, 2021

Related U.S. Application Data
(60) Provisional application No. 62/745,788, filed on Oct. 15, 2018.

(51) Int. Cl. A61K 38/20; A61K 38/18; A61K 38/19; A61K 38/21; A61K 38/30

(52) U.S. Cl. CPC A61K 38/2053; A61K 38/1866; A61K 38/1825; A61K 38/1808; A61K 38/1858; A61K 48/00; A61K 38/1816; A61K 38/191; A61K 38/217; A61K 38/193; A61K 38/30; A61K 38/1841

(57) ABSTRACT

Cell-free compositions for promoting restoration of tissue function or enhancement of tissue function and methods of stimulating or promoting restoration or enhancement of tissue function using the cell-free compositions. The compositions herein help stimulate endogenous pathways via a subject's own cells effectively for improving tissue function, enhancing tissue function, enhancing cell proliferation, etc. The compositions comprise one or a plurality of therapeutic components such as growth factors, extracellular matrix, DNA, RNA, hormones, drugs, cell surface receptors, enzymes, cytokines, angiogenesis modulating factors, etc., e.g., any material that can effectively activation endogenous pathways in the subject's own cell to a desired effect. The cell-free composition comprises a carrier or is attached to or integrated into and/or within a carrier. The carrier may help provide for containment of the therapeutic components and/or provide for time-release of the therapeutic components of the cell-free composition.

### CROSS-REFERENCES TO RELATED APPLICATIONS

This application claims benefit of U.S. Patent Application No. 62/745,788 filed Oct. 15, 2018, the specification(s) of which is/are incorporated herein in their entirety by reference.

### BACKGROUND OF THE INVENTION

### Field of the Invention

The present Invention relates to therapies for promoting tissue repair, restoration of tissue function, and/or enhancement of tissue function in a target tissue, more particularly to cell-free compositions and methods for helping promote tissue repair, restoration of tissue function, and/or enhancement of tissue function in a target tissue featuring said cell-free compositions. The compositions herein do not necessarily comprise living cells but rather comprise components produced by cells, components capable of being produced by cells, and/or components engineered or manufactured by means other than production by living cells.

### Background Art

In recent years, three-dimensional cell and/or tissue cultures have been used for various therapeutic purposes, particularly to help support tissue repair. These three-dimensional cell/tissue cultures attempt to provide conditions similar to what would be expected in vivo. It was surprisingly discovered that compositions for promoting tissue repair (or for enhancing or restoring tissue function) do not necessarily need to comprise living cells but rather can comprise components produced by cells, components capable of being produced by cells, or engineered or manufactured components. Rather than implant cells that are expected to survive and engraft in a target tissue, the compositions and methods of the present invention help stimulate tissue repair, restoration of tissue function, and/or enhancement of tissue function via stimulation of the subject's own cells (by stimulating endogenous pathways). The compositions and methods herein are not limited to use in subjects having suffered a trauma or a particular disease or condition but may also be used in healthy subjects, e.g., for enhancement of tissue function, cosmetic use, etc.

### BRIEF SUMMARY OF THE INVENTION

The present invention features cell-free compositions for promoting tissue repair, restoration of tissue function, and/or enhancement of tissue function in target tissues and methods of use. For example, the cell-free composition of the present invention may comprise a carrier and at least one stimulating agent for activating an endogenous pathway in a target tissue. When the cell-free composition is administered to a subject, the cell-free composition promotes repair of the target tissue, restoration of function of the target tissue, or enhancement of function of the target tissue. The present invention is not limited to one stimulating agent in the cell-free composition. In certain embodiments, the composition comprises two or more stimulating agents. In certain embodiments, the composition comprises three or more stimulating agents. In certain embodiments, the composition comprises four or more stimulating agents. In certain embodiments, the composition comprises five or more stimulating agents.

In certain embodiments, the stimulating agent is one or a combination of RNA, DNA, protein, carbohydrate, or a lipid. In certain embodiments, the stimulating agent is a nucleic acid.

In certain embodiments, the nucleic acid is RNA. In certain embodiments, the stimulating agent is a virus. In certain embodiments, the stimulating agent is a composition for in vivo gene editing. In certain embodiments, the stimulating agent is one or a combination of a growth factor, an angiogenesis modulating agent, a hormone, a drug, a cell surface receptor, an enzyme, a nucleic acid.

In certain embodiments, the growth factor is one or a combination of: vascular endothelial growth factor (VEGF), a fibroblast growth factor (FGF), epidermal growth factor (EGF), platelet derived growth factor (PDGF), Transforming growth factors-beta (TGF-beta), transforming growth factor-alpha (TGF-alpha), an insulin-like growth factor, erythropoietdn (Epo), tumor necrosis factor-alpha (TNF-alpha), tumor necrosis factor-beta (TNF-beta), interferon-gamma (INF-gamma), or a colony stimulating factor (CSFS). In certain embodiments, the angiogenesis modulating agent is one or more of angiopoietin-1, epidermal growth factor (EGF), hepatocyte growth factor (HGF), tumor necrosis factor (TNF-alpha), platelet derived endothelial cell growth factor (PD-ECGF), platelet derived growth factor (PDGF), insulin-like growth factor (IGF), interleukin-8 (IL-8), growth hormone (GH), angiopoietin, vascular endothelial growth factor (VEGF), a fibroblast growth factor (FGF), transforming growth factor alpha (TGF-alpha), CYR 61, or platelet-derived growth factor (PDGF).

In certain embodiments, the cell-free composition (e.g., stimulating agent) promotes production of a growth factor in the cell. In certain embodiments, the cell-free composition (e.g., stimulating agent) modifies DNA of cells in the target tissue. In certain embodiments, the cell-free composition (e.g., stimulating agent) causes gene expression of cells in the target tissue to be modified. In certain embodiments, the cell-free composition (e.g., stimulating agent) promotes or enhances cell-cell communication. In certain embodiments, the cell-free composition (e.g., stimulating agent) increases vessel density in the target tissue. In certain embodiments, the cell-free composition (e.g., stimulating agent) promotes electrical signal transduction. In certain embodiments, the cell-free composition (e.g., stimulating agent) causes activation of tissue remodeling. In certain embodiments, the cell-free composition (e.g., stimulating agent) promotes cell proliferation. In certain embodiments, the cell-free composition (e.g., stimulating agent) promotes cell differentiation. In certain embodiments, the cell-free composition (e.g., stimulating agent) promotes cell survival. In certain embodiments, the cell-free composition (e.g., stimulating agent) promotes energy production. In certain embodiments, the cell-free composition (e.g., stimulating agent) activates cell repair pathways. In certain embodiments, the cell-free composition (e.g., stimulating agent) promotes secretion of secretory factors.

The carrier may be designed in a variety of forms. For example, in certain embodiments, the carrier is a foam. In certain embodiments, the carrier is a film. In certain embodiments, the carrier is a patch. In certain embodiments, the carrier is a capsule. In certain embodiments, the carrier is a sponge. In certain embodiments, the carrier is a gel. The carrier may be designed to allow for time-release of one or more therapeutic components, e.g., one or more stimulating agents. In certain embodiments, the carrier comprises a biological matrix, a synthetic matrix, or a combination thereof. In certain embodiments, the carrier comprises a collagen matrix, gelatin, or a combination thereof. In certain embodiments, the carrier is a bulking agent. In certain embodiments, the carrier comprises proteins, gels, or a combination thereof, in certain embodiments, the carrier targets the cell-free composition to the target tissue. In certain embodiments, the carrier helps attach the cell-free composition to the target tissue. In certain embodiments, the carrier helps contain the cell-free composition in the target tissue.

Target tissues include but are not limited to muscle (e.g., skeletal muscle, heart muscle), bone, cartilage, central nervous system tissue, eye tissue, epithelial tissue, gastrointestinal tissue, auditory tissue, genital tissue, oral cavity tissue, or connective tissue, or the like.

The present invention also features methods of promoting repair, restoration of function, or enhancement of function in a target tissue of a subject in need thereof. The methods may comprise introducing to the target tissue a cell-free composition comprising according to any of the embodiments herein, wherein the cell-free composition promotes repair, restoration of function, or enhancement of function of the target tissue. The target tissue may be damaged tissue, e.g., tissue damaged from trauma, tissue damaged from disease. However, the present invention is not limited to diseased or damaged tissue. In certain embodiments, the target tissue is a healthy tissue.

Administering the cell-free composition to the target tissue may comprise Injecting the composition, e.g., Injecting a first administration of the cell-free composition. Administration is not limited to injection methods. In certain embodiments, the method further comprises administering at least one subsequent administration of a cell-free composition according to any of the embodiments herein.

The present invention also features methods of promoting repair, restoration of function, or enhancement of function in gastrointestinal tissue of a subject in need thereof. The methods may comprise introducing to the target tissue a cell-free composition comprising according to any of the embodiments herein, wherein the cell-free composition promotes repair, restoration of function, or enhancement of function of the gastrointestinal tissue. In certain embodiments, the cell-free composition causes the gastrointestinal tissue to increase production of enzymes, the cell-free composition causes the gastrointestinal tissue to increase motility.

The present invention also features methods of promoting repair, restoration of function, or enhancement of function in cardiac tissue of a subject in need thereof. The methods may comprise introducing to the target tissue a cell-free composition comprising according to any of the embodiments herein, wherein the cell-free composition promotes repair, restoration of function, or enhancement of function of the liver tissue. In certain embodiments, the cell-free composition causes the cardiac tissue to restore blood flow. In certain embodiments, the cell-free composition causes an Increase in electrical function of cardiac tissue.

Any feature or combination of features described herein are Included within the scope of the present invention provided that the features included in any such combination are not mutually inconsistent as will be apparent from the context, this specification, and the knowledge of one of ordinary skill in the art. Additional advantages and aspects of the present Invention are apparent in the following detailed description and claims.

### Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly Indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example. Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel st al., Current Protocols in Molecular Biology, Greene Publishing Associates, 1992 (and Supplements to 2000): Ausubel et al., Short Protocols in Molecular Biology: A Compendium of Methods from Current Protocols in Molecular Biology, 4th ed., Wiley & Sons, 1999: Harlow and Lane, Antbodies: A Laboratory Manual, Cold Spring Harbor Laboratory Press, 1990; and Harlow and Lane. Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ad., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss. Inc. New York, N.Y.), Gene Transfer and Expression Protocols, pp. 109-128, ad. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

The term “angiogenesis modulating agent” as used herein refers to any agent that can induce angiogenesis or the proliferation of endothelial cells. For example, an angiogenesis modulating agent may include a cytokine (e.g., interleukins, lymphokines, monokines, interferons, colony stimulating agents, chemokines, other proteins, etc.), a growth factor, an enzyme, an enzymatic inhibitor, an antibody, etc. One class of angiogenesis modulating agents are polypeptide angiogenic factors, which includes but is not limited to angiopoietin-1, epidermal growth factor (EGF), hepatocyte growth factor (HGF), tumor necrosis factor (TNF-alpha), platelet derived endothelial cell growth factor (PD-ECGF), platelet derived growth factor (PDGF), insulin-like growth factor (IGF), interleukin-8, growth hormone, angiopoietin, vascular endothelial growth factor (VEGF), acidic and basic fibroblast growth factors (FGFs), transforming growth factor alpha (TGF-alpha), CYR 61, platelet-derived growth factor (PDGF), etc. These molecules have been shown to induce angiogenesis in vivo. Other similar molecules that display angiogenic activity are the heparin binding growth factors (HBGFs). Other angiogenesis modulating agents have been described in addition to polypeptide angiogenic factors. For example, prostaglandins E1 and E2, which are lipid-derived angiogenic factors, are well known inflammatory cell attractants with angiogenic properties. In addition, nicotinamide causes an angiogenic response when tested in chick cornea or in a chick CAM assay. In addition, negative angiogenic regulatory molecules include angiostatin; endostatin; and thrombospondin. The present Invention is not limited to the aforementioned angiogenesis modulating agents.

The term “derived from” as applied to a cellular component being “derived from” a cell means the component was produced from a cell and isolated from said cell. The term “derived from” as applied to a cell being “derived from” another cell or from a tissue means the cell was either Isolated from the tissue referred to, or was differentiated from the reference tissue or cell type.

The term “disease” or “disorder” or “condition” refers to any alteration in state of the body or of some of the organs, interrupting or disturbing the performance of their functions and/or causing symptoms such as discomfort, dysfunction, distress, or even death to the person afflicted or those in contact with a person. A disease or disorder or condition can also relate to a distemper, ailing, ailment, malady, disorder, sickness, illness, complaint, indisposition or affliction.

The term “growth factors” refers to substances that promote cell growth. Growth factors are also known to stimulate cell migration (e.g., mitogenic cytokines), function as chemotactic agents, inhibit cell migration or invasion of tumor cells, modulate differentiated functions of cells, be Involved in apoptosis and promote survival of cells. Such factors can be secreted as diffusible factors and can also exist in membrane-anchored forms. They can, therefore, act in an autocrine, paracrine, juxtacrine or retrocrine manner. A cytokine is one type of growth factor. Examples of growth factors include, but are not limited to, VEGF, fibroblast growth factors (FGF), epidermal growth factor (EGF), platelet derived growth factor (PDGF). Transforming growth factors-beta (TGF-beta), transforming growth factor-alpha (TGF-alpha), insulin-like growth factors, erythropoietin (EPO), tumor necrosis factor-alpha (TNF-alpha), tumor necrosis factor-beta (TNF-beta), interferon-gamma (INF-gamma), and colony stimulating agents (CSFS) or variants thereof. The present invention is not limited to the aforementioned growth factors or the others described herein.

As used herein, “proliferating” and “proliferation” refers to an Increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “scaffold” refers to a support structure for therapeutic material. The support structure may feature fibers, the scaffold may be in the form of a film, sponge, solution, etc. The scaffold may be constructed from a variety of materials such as but not limited to fibers, peptides (e.g., recombinant peptides), lipids, carbohydrates, etc.

The term “stimulating agent(s)” as used herein refers to any molecule or composition of the cell-free composition that has an effect on a pathway of an endogenous cell in the subject being administered a cell-free composition of the present invention. Stimulating agents are not limited to activators of pathways; a stimulating agent may activate a particular pathway and/or inhibit a particular pathway. Non-limiting examples of stimulating agents include: DNA, RNA, miRNA, a peptide/protein, a virus, a carbohydrate, a lipid, a drug, a composition comprising one or a combination of the aforementioned molecules, etc. For example, a stimulating agent may be a miRNA, a virus, a growth factor, a hormone, a cytokine, an angiogenesis modulating agent, a drug, a cell-surface receptor, an antibody or fragment thereof, etc. The present invention is not limited to the aforementioned examples of stimulating agents described herein.

The term “tissue” refers to a group or layer of similarly specialized cells that together perform certain special functions.

As used herein, the terms “treat” or “treatment” or “treating” refers to both therapeutic treatment and prophylactic or preventative measures or enhancement measures (e.g., enhancement of function of healthy tissue), wherein the object is to prevent or slow the development of the disease, such as slow down the development of a cardiac disorder, or reducing at least one adverse effect or symptom of a cardiovascular condition, disease or disorder, e.g., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, a treatment is “effective” If the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival, restoring function, or enhancing function as compared to expected survival or function if not receiving treatment. Those in need of treatment include those already diagnosed with a condition, as well as those likely to develop a condition due to genetic susceptibility or other factors such as weight, diet and health, as well as healthy individuals.

### DETAILED DESCRIPTION OF THE INVENTION

The present invention features cell-free compositions for restoration of tissue function or enhancement of tissue function in target tissues, and methods of use. The compositions herein help stimulate endogenous pathways via a subject's own cells effectively for improving tissue function, enhancing tissue function, enhancing cell proliferation, etc. Briefly, the compositions comprise one or a plurality of therapeutic components functioning as stimulating agents, e.g., molecules, compound, or compositions that affect pathways of endogenous cells to stimulate restoration of tissue function or enhancement of tissue function in a particular target tissue. Non-limiting examples of stimulating agents Include growth factors, extracellular matrix components, protein, DNA, RNA, hormones, drugs, cell surface receptors, enzymes, cytokines, angiogenesis modulating factors, etc., e.g., any material that can appropriately affect (e.g., activate, inhibit) particular endogenous pathways in the subject's own cell to achieve a desired effect. As an example, a stimulating agent from the cell-free composition may lead to the activation of pathways in the subject's own cells that lead to secretion of growth factor so as to promote cell survival, cell repair, cell function, regain of tissue function, or enhancement of tissue function.

Subjects include but are not limited to humans or other primates or any other appropriate animal model such as mice, rats, pigs, cats, dogs, rabbits, etc.

Non-limiting examples of target tissues include muscle (e.g., skeletal muscle, cardiac muscle, etc.), bone, cartilage, central nervous system tissue (e.g., nerves, myelin sheath, etc.), eye tissue, epithelial tissue, gastrointestinal tissue (e.g., colon tissue, pancreas tissue, liver tissue, etc.), ear and auditory tissue, genital tissue, oral cavity tissue (e.g., salivary tissue, dental tissue, etc.), connective tissue, etc.

The exact combination of components, e.g., therapeutic components, stimulating agents, used to produce the cell-free compositions of the present invention may vary depending on the target tissue. Different tissues may have different requirements for repair, restoration of function (e.g., restoration of cell-cell communication, immune response, extracellular matrix production, etc.), etc. As an example, the restoration of function of heart muscle may involve remodeling and restoration of cellular communication for coordinated electrical signaling and mechanical force generation. As an example, the effect on heart tissue may include agents to enhance survival of endogenous cells, enhance function of endogenous cells, Including but not limited to cardiomyocytes, as well as agents that promote proliferation of endogenous cells, including but not limited to fibroblasts, endothelial cells, etc. Further, the combination of components used to produce the cell-free compositions of the present invention will also vary depending on the underlying cause of the tissue damage, e.g., whether the damage was a result of trauma, a chronic disease, a congenital disease, a genetic abnormality, etc., or whether the composition is used in a healthy subject for the purpose of enhancing tissue function (e.g., enhancing muscle strength, endurance, etc.).

In certain embodiments, the cell-free compositions of the present invention comprise material produced by cells. In certain embodiments, the cell-free compositions of the present invention comprise material capable of being produced by cells. In certain embodiments, the cell-free compositions of the present invention comprise material that is engineered or manufactured.

Non-limiting examples of therapeutic components or stimulating agents of the cell-free compositions of the present invention include cell surface receptors from intact livings cells as well as non-living or non-living and non-intact cells, e.g. toll-like receptors (TLRs), CD40, growth factor(s); DNA; RNA (e.g., RNA, miRNA, etc.); cytokines (e.g., interferon gamma, interleukins (e.g., IL-4, IL-5, IL-8, IL-13, IL-17, IL-22, etc.); viruses, RNAs including but not limited to microRNAs; hormones; drugs; extracellular matrix; proteins, proteasomes, and glycoproteins, e.g. major basic protein (MBP), granule proteins, heat shock proteins (HSPs), granulocyte colony-stimulating factor, granulocyte-macrophage colony stimulating factor; proteases; glycans, proteoglycans; chemokines (e.g., MCP-1, macrophage inflammatory proteins (MIPs), RANTES, IP-10); vasoactive agents; angiogenesis modulating agents; enzymes (e.g. MMPs, elastase, cathepsins, lysosomal hydrolytic enzymes, eosinophilic peroxidase, proteases); gene editing compositions (e.g., one or more components for the purpose of in vivo gene editing); tryptase, chymase; other biologically active components; gene therapies, e.g., gene therapies via viruses, RNA, DNA, etc.; the like, or a combination thereof.

Growth factors may include but are not limited to: vascular endothelial growth factor (VEGF), fibroblast growth factor (FGF), connective tissue growth factor (CTGF), basic fibroblast growth factor (bFGF), hepatocyte growth factor (HGF), angiopoietin-1, matrix deposit factors (e.g., Transforming growth factors, e.g., TGF-a, TGF-b, e.g., TGF-b1, TBG-b3, etc.), mitogenic factors (e.g., platelet derived growth factor (PDGF, e.g., platelet derived growth factor A (PDGF-A), Insulin like growth factor 1 (IGF-1), Heparin binding epidermal growth factor (HBEGF), Transforming growth factor a (TGFa)), angiogenic factors (e.g., Angiogenein, Angiopoientin-2), Endothelial Growth Factor, Leptin, Platelet derived growth factor BB (PDGF-BB), Vascular endothelial growth factor (VEGF), Hepatocyte growth factor (HGF), Basic Fibroblasts growth factor (bFGF), Secreted protein acid and rich in cysteine (SPARC), Interleukin 6 (IL-6), interleukin 8 (IL-8), Inflammatory Cytokines (e.g., Interferon-gamma, Interleukin 1a, Interleukin 1b, Interleukin 6 (IL-6), interleukin 8 (IL-8), Monocyte chemotactic protein 1, Granulocyte colony stimulating factor (GCSF), Tumor necrosis factor a (TNFa), EPO, etc.

The cell-free compositions herein comprise a carrier. For example, the compositions may be attached to a carrier, integrated into a carrier, encapsulated by a carrier, etc. The carrier may help provide for containment of the therapeutic components and/or targeting or localization of the therapeutic components of the composition and/or provide for time-release of the therapeutic components of the cell-free composition. For example, the carrier could be used for targeted delivery of the therapeutic components (e.g., the stimulating factor(s)), to avoid off-target affects, to help minimize the amount of therapeutic agent needed for effectiveness (e.g., reduce waste, reduce cost, etc.). Targeting is not limited to specific targeting wherein the carrier comprises a receptor that is specific for a ligand of the target tissue or vice versa. Targeting may refer to merely the physical administration of the composition to the location needed. Targeting may refer to physical contact with the intended target tissue. Targeting may refer to containment of the composition in the area of the target tissue, etc.

Carriers (e.g., pharmaceutical carriers, pharmaceutical vehicles, pharmaceutical compositions, pharmaceutical molecules, etc.) are materials generally known to deliver cells and/or drugs and/or other appropriate material into the body. The compositions herein may feature a carrier, which may function as a preservative agent, a timed-release control agent, a bulking agent, etc. for the components of the composition. For example, a carrier may comprise a patch, e.g., a material in which components of the cell-free composition are adsorbed for slow release or encapsulated for slow/sustained, fast/acute or controlled release. In certain embodiments, components of the cell-free composition may be physically encapsulated or biochemically bound by a carrier. Carrier materials may be treated to enhance encapsulation or binding activity of the components of the cell-free composition.

Carriers (e.g., pharmaceutical carriers or vehicles) may comprise conventional carriers but are not limited to conventional carriers. For example, E. W. Martin, Remington's Pharmaceutical Sciences, Mack Publishing Co., Easton. Pa., 15th Edition (1975) and D. B. Troy, ed. Remington: The Science and Practice of Pharmacy, Lippincott Williams & Wilkins, Baltimore Md. and Philadelphia, Pa., 21^{st} Edition (2006) describe compositions and formulations suitable for pharmaceutical delivery of one or more therapeutic compounds or molecules. In general, the nature of the carrier will depend on the nature of the composition being delivered as well as the particular mode of administration being employed. In addition to biologically-neutral carriers, pharmaceutical compositions administered may contain minor amounts of non-toxic auxiliary substances, such as wetting or emulsifying agents, preservatives, and pH buffering agents and the like. Patents that describe pharmaceutical carriers include, but are not limited to: U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,948,931, the disclosures of which are incorporated in their entirety by reference herein.

The carrier may, for example, be solid, liquid (e.g., a solution), foam, a gel, the like, or a combination thereof. In some embodiments, the carrier comprises a biological matrix (e.g., biological fibers, etc.). In some embodiments, the carrier comprises a synthetic matrix (e.g., synthetic fibers, etc.). In certain embodiments, a portion of the carrier may comprise biological matrix and a portion may comprise synthetic matrix.

In certain embodiments, the carrier comprises a collagen matrix, gelatin, the like, or a combination thereof. In certain embodiments, the carrier may resemble a scaffold, e.g., a composition providing bulk, thickness, and/or structural support for components of the cell-free composition. Non-limiting examples of materials that may commonly be used for constructing scaffolds include: polyglycolide, polylactide, polyhydroxobutyrate, poly(anhydrdes), poly(dioxanone), poly (trimethylene carbonate), polyglactin, poly(lactic acid), polyvinylidene fluoride, polyesters, silicone, polyurethane, polymethylmethacrylate, polypropylene, polyethylene, poliglecaprone-25 monofilament, a polycarbonate, a polyamide, a polyesters, a polystyrene, a polyacrylate, a polyvinyl, polytetrafluorethylene, thermanox, nitrocellulose, gelatin, dextran, collagen, fibrin, elastin, silk, metals. TMC, proteins/peptides, the like, or a combination thereof. In certain embodiments, the carrier or scaffold is in the form of a film. In certain embodiments, the carrier or scaffold is a sponge. In certain embodiments, the carrier or scaffold is stratified having multiple levels or layers.

Non-limiting examples of carriers include hydrogels (e.g. collagen, gelatin, fibrin, glycosaminoglycans, the like, or combinations thereof); tissues; porous scaffolds; non-porous scaffolds; synthetic scaffolds (e.g., poly(lactic acid), poly(glycolic acid), poly(caprolactone), co-polymers, the like, combinations thereof); natural scaffolds; scaffolds from a combination of synthetic or natural materials; ceramics; composites; natural polymers (e.g. collagen, fibrin, silk, glycosaminoglycans, the like, or a combination thereof); liposomes; micelles; microspheres; nanoparticles or nanocapsules or other nanostructures such as nanoshells (optionally allowing for controlled release); proteins; fatty acids; carbohydrates, gels; etc.

The carrier may be absorbable or non-absorbable. Or, the carrier may comprise a portion that is absorbable and a portion that is non-absorbable. In some embodiments, a portion of the carrier resorbs/absorbs/degrades within 1 day of administration (e.g., injection, implantation, etc.). In some embodiments, a portion of the carrier resorbs/absorbs/degrades within 1 week of administration (e.g., injection, implantation, etc.). In some embodiments, a portion of the carrier resorbs/absorbs/degrades within 2 weeks administration (e.g., injection, implantation, etc.). In some embodiments, a portion of the carrier resorbs/absorbs/degrades within 1 month of administration (e.g., injection, implantation, etc.). In some embodiments, a portion of the carrier resorbs/absorbs/degrades within 6 months of administration (e.g., injection, implantation, etc.). In some embodiments, a portion of the carrier resorbs/absorbs/degrades within 1 year of administration (e.g., injection, implantation, etc.). In some embodiments, a portion of the carrier resorbs/absorbs/degrades within 2 years of administration (e.g., injection, Implantation, etc.). In some embodiments, a portion of the carrier resorbs/absorbs/degrades within 3 years of administration (e.g., injection, implantation, etc.). In some embodiments, a portion of the carrier resorbs/absorbs/degrades within 4 years of administration (e.g., injection, implantation, etc.). Any appropriate degradable materials or combinations thereof may achieve a desired degradation profile.

The carrier or scaffold may be anisotropic, e.g., the mechanical properties of the carrier or scaffold may be different in one direction than the other. In certain embodiments, the carrier or scaffold may be isotropic.

The carrier may comprise components that are products of cells. For example, in certain embodiments, the product is extracellular matrix (ECM). The ECM may be derived from any appropriate ECM-generating cells. ECM components may include but are not limited to collagen (e.g., collagen type I, collagen Type II), elastin, fibronectin, laminins, tenascin, proteoglycans, glycosaminoglycans (e.g., Veriscan, Decorin, Betaglycan, Syndecan), etc.

In some embodiments, the carrier itself further comprises one or more growth factors. Growth factors may include but are not limited to vascular endothelial growth factor (VEGF), basic fibroblast growth factor (bFGF), hepatocyte growth factor (HGF), angiopoietin-1, matrix deposit factors (e.g., Transforming growth factor (TGF-b1), Transforming growth factor (TBG-b3)), mitogenic factors (e.g., Platelet derived growth factor A (PDGF-A), Insulin like growth factor 1 (IGF-1). Heparin binding epidermal growth factor (HBEGF), Transforming growth factor a (TGFa)), angiogenic factors (e.g., Angiogenein, Angiopoietin-2), Endothelial Growth Factor, Leptin, Platelet derived growth factor BB (PDGF-BB), Vascular endothelial growth factor (VEGF), Hepatocyte growth factor (HGF), Basic Fibroblasts growth factor (bFGF), Secreted protein acid and rich in cysteine (SPARC), interleukin 6 (IL-6), Interleukin 8 (IL-8), Inflammatory Cytokines (e.g., Interferon-gamma, Interleukin 1a. Interleukin 1b, Interleukin 6 (IL-6), interleukin 8 (IL-8), Monocyte chemotactic protein 1, Granulocyte colony stimulating factor (GCSF), Tumor necrosis factor a (TNFa)), FGF, EPO, etc. The carrier may comprise any of the aforementioned proteins or agents herein.

In some embodiments, the carrier is constructed from or comprises a component for enhancing adherence to the endogenous cells of the subject. In some embodiments, the carrier is constructed from or comprises a component for enhancing adherence of the carrier to the therapeutic components of the cell-free composition. For example, in some embodiments, the carrier has a hydrophilicity adapted to allow adherence of endogenous cells of the subject to itself.

Mechanisms of release of components of the cell-free composition (e.g., therapeutic components) from the carrier include but are not limited to: hydrolysis, enzymatic degradation (e.g. collagenase); temperature sensitive changes in structure or porosity; melting; dissolution; light activated changes in structure or porosity (e.g. using resonance); electrically activated changes in structure or porosity; bioactivation (e.g. conversion to an enzyme); digestion; ultrasonic disruption: physical disruption; denaturation; biodegradation; structural changes through binding receptors or molecules; structure changes through polymerization; swelling; the like, or a combination thereof.

In certain embodiments, the cell-free composition and/or carrier is designed to allow for confirmation that the cell-free composition reaches its target or intended destination.

Table 1 below shows non-limiting examples of cell-free compositions of the present invention. The present invention Is not limited to the examples described herein.

TABLE 1
Examples of Cell-Free Compositions

| | | | | | | Components (e.g., therapeutic components) | | | | | |
|---|---|---|---|---|---|---|---|---|---|---|---|
| | | | | | | | | | | Nucleic Acid | |
| | | | | | | | | | | (e.g., RNA) | |
| | Carrier | | | | | | | Cell- | Angiogenesis | or Gene | |
| | Flim | | | Time- | Foam | Growth | | surface | Modulating | Editing | |
| Example | or Patch | Capsule | Sponge | release | or Gel | Factors | ECM | receptors | Agents | Material | Drugs |
| 1 | ✓ | | | | | ✓ | ✓ | | | | |
| 2 | ✓ | | | | | ✓ | ✓ | | ✓ | | |
| 3 | ✓ | | | | | ✓ | ✓ | | ✓ | | ✓ |
| 4 | ✓ | | | ✓ | | ✓ | ✓ | | | | |
| 5 | ✓ | | | ✓ | | ✓ | ✓ | | ✓ | | |
| 6 | ✓ | | | ✓ | | ✓ | ✓ | ✓ | | ✓ | ✓ |
| 7 | | ✓ | | | | ✓ | ✓ | | | | |
| 8 | | ✓ | | | | ✓ | ✓ | | ✓ | | |
| 9 | | ✓ | | | | ✓ | ✓ | | ✓ | | ✓ |
| 10 | | ✓ | | ✓ | | ✓ | ✓ | | | | |
| 11 | | ✓ | | ✓ | | ✓ | ✓ | | ✓ | | |
| 12 | | ✓ | | ✓ | | ✓ | ✓ | ✓ | | ✓ | ✓ |
| 13 | | | ✓ | | | ✓ | ✓ | | | | |
| 14 | | | ✓ | | | ✓ | ✓ | | ✓ | | |
| 15 | | | ✓ | | | ✓ | ✓ | | ✓ | | ✓ |
| 16 | | | ✓ | ✓ | | ✓ | ✓ | | | | |
| 17 | | | ✓ | ✓ | | ✓ | ✓ | | ✓ | | |
| 18 | | | ✓ | ✓ | | ✓ | ✓ | ✓ | | ✓ | ✓ |
| 19 | | | | | ✓ | | ✓ | | | ✓ | |
| 20 | | | | | ✓ | ✓ | | | ✓ | ✓ | |
| 21 | | | | | ✓ | ✓ | ✓ | | ✓ | | ✓ |
| 22 | | | | ✓ | ✓ | | ✓ | | | ✓ | |
| 23 | | | | ✓ | ✓ | ✓ | ✓ | | ✓ | | |
| 24 | | | | ✓ | ✓ | ✓ | ✓ | ✓ | | ✓ | ✓ |
| 25 | ✓ | | | | | | | | | ✓ | |
| 26 | | ✓ | | | | | | | | ✓ | |
| 27 | | | ✓ | | | | | | | ✓ | |
| 28 | | | | ✓ | | | | | | ✓ | |
| 29 | | | | | ✓ | | | | | ✓ | |
| 30 | | ✓ | | | | | | | ✓ | ✓ | |

### Methods of Use

The present invention features methods of restoring function a target tissue in a subject in need thereof, as well as methods of enhancing function of a target tissue in a subject in need thereof. The methods may generally comprise administering to the subject (e.g., administering specifically the target tissue) a cell-free composition of the present invention, wherein the cell-free composition promotes tissue repair, restores tissue function, and/or enhances tissue function of the target tissue.

As previously discussed, the compositions comprise one or a plurality of therapeutic components functioning as stimulating agents, e.g., molecules, RNA, compound, or compositions that affect pathways of endogenous cells to stimulate restoration of tissue function or enhancement of tissue function in a particular target tissue. The particular therapeutic components of the composition depend on the target tissue (e.g., cell-free compositions designed for cardiac tissue will differ from cell-free compositions for skin tissue) and the goal effect (e.g. survival of endogenous cells, proliferation of endogenous cells, enhanced function of endogenous cells). Likewise, the effects of the cell-free composition (e.g., the stimulating agent/agents of the composition) will activate and/or inhibit different pathways in different tissues.

In certain embodiments, the cell-free composition (e.g., stimulating agent from the cell-free composition) causes the activation of pathways in the subject's own cells that lead to secretion of growth factor so as to promote tissue repair, regain of tissue function, or enhancement of tissue function. In certain embodiments, the cell-free composition (e.g., stimulating agent from the cell-free composition) causes the subject's cells to activate or promote angiogenesis (increase vascular density) and increase blood flow to the target tissue. In certain embodiments, the cell-free composition (e.g., stimulating agent from the cell-free composition) causes the subject's cells to activate tissue remodeling. In certain embodiments, the cell-free composition (e.g., stimulating agent from the cell-free composition) causes the subject's cells to promote cell proliferation. In certain embodiments, the cell-free composition (e.g., stimulating agent from the cell-free composition) causes the subject's cells activate or promote cell differentiation. In certain embodiments, the cell-free composition (e.g., stimulating agent from the cell-free composition) causes the subject's cells to promote or enhance cell-cell communication. In certain embodiments, the cell-free composition (e.g., stimulating agent from the cell-free composition) causes the subject's cells to promote or enhance cell survival. In certain embodiments, the cell-free composition (e.g., stimulating agent from the ceil-free composition) causes the subject's cells to promote or enhance energy production. In certain embodiments, the cell-free composition (e.g., stimulating agent from the cell-free composition) causes the subject's cells to promote or enhance protein production. In certain embodiments, the cell-free composition (e.g., stimulating agent from the cell-free composition) causes the subject's cells to promote or enhance RNA production. In certain embodiments, the cell-free composition (e.g., stimulating agent from the cell-free composition) causes the subject's cells to promote or enhance cell death. In certain embodiments, the cell-free composition (e.g., stimulating agent from the cell-free composition) causes the subject's cells to inhibit cell death. In certain embodiments, the cell-free composition (e.g., stimulating agent from the cell-free composition) causes the subject's cells to activate cell repair pathways. In certain embodiments, the cell-free composition (e.g., stimulating agent from the cell-free composition) causes the subject's cells to promote or enhance secretion of particular secretory factors. In certain embodiments, the cell-free composition (e.g., stimulating agent from the cell-free composition) causes the subject's cells to promote or enhance electrical signal transduction.

In certain embodiments, the cell-free composition (e.g., stimulating agent from the cell-free composition) causes the DNA In the subject's cells to be modified. In certain embodiments, the cell-free composition (e.g., stimulating agent from the cell-free composition) causes gene expression in the subject's cells to be modified. In certain embodiments, the cell-free composition (e.g., stimulating agent from the cell-free composition) causes the subject's cells to release a particular hormone. The present invention is not limited to the aforementioned pathways that may be affected by the cell-free compositions herein.

The cell-free compositions herein can increase vascular density in a target tissue. In certain embodiments, the composition increases vascular density in a target tissue by 1-fold or more. In certain embodiments, the composition increases vascular density in a target tissue by 2-fold or more. In certain embodiments, the composition increases vascular density in a target tissue by 3-fold or more. In certain embodiments, the composition increases vascular density in a target tissue by 4-fold or more. In certain embodiments, the composition increases vascular density in a target tissue by 5-fold or more.

The cell-free compositions can increase blood flow in the target tissue. In certain embodiments, the composition Increases blood flow in a target tissue by 1-fold or more. In certain embodiments, the composition increases blood flow in a target tissue by 2-fold or more. In certain embodiments, the composition increases blood flow in a target tissue by 3-fold or more. In certain embodiments, the composition increases blood flow in a target tissue by 4-fold or more. In certain embodiments, the composition increases blood flow in a target tissue by 5-fold or more.

Restoration or enhancement of tissue function also depends on the particular target tissue. For example, restoration or enhancement of liver tissue function may refer to an increase in hepatic secretions. Restoration or enhancement of cardiac tissue function may refer to an increase in electrical function of cells, restoration of cell-cell communication between cells, etc. For example, restoration of cardiac tissue function may refer to Improved contractility of stunned or hibernating myocardium, improved muscle contractions in an akinetic or hypokinetic heart, etc. Restoration of cardiac tissue may even refer to the inhibition of disease progression, stabilization of the heart, etc. Restoration of skin tissue may refer to regeneration of skin after a significant burn. Restoration or enhancement of auditory tissue may refer to increased function of the cochlea, e.g., improved hearing.

Methods of administration of the compositions of the present Invention may include injection, implantation, or oral administration.

The cell-free composition may be delivered under the visceral layer of a particular tissue or organ. In certain embodiments, the cell-free composition may be delivered under the parietal layer of a particular organ or tissue, e.g., the space between the parietal layer and the visceral layer. The target for the cell-free composition may be accessed through both the visceral and parietal layers, e.g., the two layers may be dissected to provide access to the tissue or organ. For example, the composition may be delivered to the pleural space, e.g., the space between the visceral pleura and the parietal pleura. The composition may be delivered to the heart tissue below the visceral pericardium. The present invention is not limited to the aforementioned examples of specific delivery of the cell-free compositions.

Administration of the cell-free compositions of the present invention may feature a single administration. In certain embodiments, administration of the cell-free compositions may feature two or more administrations of the cell-free composition (e.g., two or more administrations of the same composition, two or more administrations of different compositions, a combination thereof, etc.).

As an example, in applications related to cardiac tissue, one or a combination of cell-free compositions of the present invention may be administered (e.g., injected) into one or more locations such as: the myocardium, epicardium and/or endocardium of the left ventricle, right ventricle, septum, under the parietal pericardium, between the parietal pericardium and visceral pericardium, and/or under the visceral pericardium, etc. The administrations (e.g., injections) may be performed in a particular directional order, e.g., the administrations may start from the peri-infarct (border) zone toward the infarct zone (the dosing and spacing of injections may depend on the size of the diseased area and severity of disease). The procedure may be a single administration. Or, in certain embodiments, the procedure may feature a series of administrations, e.g., 2, 3, 4, 5, from 1 to 10, etc. Administrations may be spaced as appropriate, e.g., days apart (e.g., 1 day, 2 days, 3 days, 4 days, 5 days, 6 days, 7 days, 10 days, 15 days, 20 days, from 7 to 21 days, etc.), weeks apart (e.g., 1 week apart, 2 weeks apart. 3 weeks apart, 4 weeks apart, 5 weeks apart, etc.), etc.

As an example, in applications related to muscle tissue, one or a combination of cell-free compositions of the present Invention may be administered (e.g., injected) into one or more target locations (e.g., in and/or around the target muscle). The administrations (e.g., injections) may be performed in a particular directional order. In certain embodiments, the administrations (e.g., injections) may be performed as a stand-alone procedure. In certain embodiments, the administrations (e.g., injections) may be performed as adjunct to another procedure. The procedure may be a single administration. Or, in certain embodiments, the procedure may feature a series of administrations, e.g., 2, 3, 4, 5, from 5 to 10, from 10 to 20, etc. Administrations may be spaced as appropriate, e.g., days apart (e.g., 1 day, 2 days, 3 days, 4 days, 5 days, 8 days, 7 days, 10 days, 15 days, 20 days, from 7 to 21 days, etc.), weeks apart (e.g., 1 week apart, 2 weeks apart, 3 weeks apart, 4 weeks apart, 5 weeks apart, etc.), months apart (e.g. 1 month, 2 months, 6 months, etc.), etc.

The cell-free compositions of the present invention are constructed to withstand short-term and/or long-term storage, e.g., cryopreservation. Cryopreservation may refer to a temperature of −80° C. to −196° C. or −90° C. to 196° C. The ability to cryopreserve the composition helps allow for stocking until use, as well as for transporting the compositions from one location to another.

The cell-free compositions may also be constructed to withstand certain lengths of time at room temperature (or a temperature below 37° C.). Without wishing to limit the present invention to any theory or mechanism, it is believed that the ability to remain functional or usable at a temperature below 37° C. for certain lengths of time may be beneficial for instances when the composition is out of the incubator prior to use. As an example, a composition may be exposed to room temperature for a lengthy period of time when it is removed from the incubator, brought to a room for administration, but is not administered immediately.

### Example 1

Example 1 describes data obtained from tissue cultures of fibroblast only cultures (Table 2), co-cultured cardiac grafts (cardiomyocytes and fibroblasts) (Table 2, Table 3), and grafts containing cardiac progenitor cells and fibroblasts (Table 2). Various cytokines, proteins, growth factors. etc. were given off by these cells, resulting in tissue repair/associated functional benefit.

Table 2 below shows gene expression of cardiac graft cultures of terminally differentiated cardiomyocytes co-cultured with fibroblasts and of cardiac progenitor cells co-cultured with fibroblasts (vascular endothelial growth factor (VEGF), stromal derived factor-1 (SDF-1a), Insulin like growth factor-1 (IgF-1), interleukin-6 (IL-6), IL-8, and IL-10). N=2.

TABLE 2

| | Terminally differentiated | |
|---|---|---|
| | cardiomyocytes co- | Cardiac progenitor cells co- |
| | cultured with fibroblasts | cultured with fibroblasts |
| Agent | (fold expression) | (fold expression) |
| VEGF | 8 | 7 |
| SDF-1 alpha | 5 | 2 |
| IgF-1 | 8 | 7 |
| IL-6 | 12 | 12 |
| IL-8 | 9 | 10 |
| IL-10 | 8 | 6 |

Table 3 below shows growth factor and cytokine expression levels of fresh vs thawed (previously cryopreserved) cardiac grafts (terminally differentiated cardiomyocytes co-cultured with fibroblasts) as assessed through ELISA assay. Data are mean+SE. * denotes statistical difference (p<0.05). N=3 for Fresh, n=3 for Thawed.

TABLE 3

| Agent | Fresh (pg/ml) | Thawed (pg/ml) |
|---|---|---|
| IGF-1 | 0.16 | 0.17 |
| VEGF | 1300 | 800* |
| FGF | 30 | 70 |
| HGF | 650 | 1200* |
| G-CSF | 1200 | 1600 |
| TGF-b | 750 | 800 |

Table 4 below shows cytokine, chemokine and growth factor stimulation of angiogenic and inflammatory markers in static (control) versus strained fibroblasts grafts in vivo for over 48 hrs (10% 1 Hz). Expression is in pg cytokine/ug DNA, n=3. Adjusted for 2% fetal bovine serum in medium. *p<0.05.

TABLE 4
Cytokine Activation in Static (control)
versus Strained Fibroblast Grafts

| | Static | Strained |
|---|---|---|
| Angiogenic Agents | | |
| Angiogenin | 1322 ± 769 | 383 ± 140 |
| Angiopoientin-2 | −71 ± 183 | −180 ± 100 |
| Basic Fibroblast Growth Factor | 461 ± 290 | 38 ± 50 |
| Endothelial Growth Factor | 15 ± 0.7 | 5 ± 3 |
| Heparin-Binding Endothelial | −226 ± 189 | −172 ± 49 |
| Growth Factor | | |
| Hepatocyte Growth Factor | 9725 ± 4307 | 9285 ± 5219 |
| Leptin | −152 ± 3610 | 459 ± 1970 |
| Platelet-Derived Growth Factor BB | 38 ± 8 | 23 ± 4 |
| Placental Growth Factor | 11 ± 4 | 27 ± 14 |
| Vascular Endothelial Growth Factor | 324 ± 152 | 758 ± 392 |
| Inflammation Factors | | |
| Interferon-gamma | 3573 ± 258 | 2060 ± 153* |
| Interleukin-1α | 577 ± 23 | 212 ± 6* |
| Interleukin-1β | 171 ± 27 | 36 ± 5* |
| Interleukin-4 | 101 ± 16 | 36 ± 7* |
| Interleukin-6 | 83 ± 33 | 36 ± 19 |
| Interleukin-8 | 3937 ± 2192 | 2966 ± 1517 |
| Interleukin-10 | −20 ± 14 | −48 ± 8 |
| Interieukin-13 | −77 ± 36 | −137 ± 34 |
| Monocyte Chemotactic Protein-1 | 11900 ± 5890 | 10830 ± 2847 |
| Tumor Necrosis Factor α | 2183 ± 100 | 1277 ± 110* |

The disclosures of the following U.S. patents are incorporated in their entirety by reference herein: U.S. Pat. App. No. 2018/0361025; U.S. Pat. No. 4,963,409; U.S. Pat. App. No. US2009/0269316; WO2013151755; WO2011102991; U.S. Pat. App. No. 2014/0178450: U.S. Pat. No. 8,802,144; WO2009102967; U.S. Pat. No. 9,119,831; WO2010042856; U.S. Pat. No. 2008/075750. U.S. Pat. No. 9,587,222.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present Invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

1. A cell-free composition comprising:
a. a carrier; and
b. at least one stimulating agent for activating an endogenous pathway in a target tissue;
wherein the cell-free composition, when administered to a subject, promotes repair of the target tissue, restoration of function of the target tissue, or enhancement of function of the target tissue.

2. The cell-free composition of claim 1, wherein the stimulating agent is one or a combination of RNA, DNA, protein, carbohydrate, or a lipid.

3.-4. (canceled)

5. The cell-free composition of claim 1, wherein the stimulating agent is a virus.

6. The cell-free composition of claim 1, wherein the stimulating agent is a composition for in vivo gene editing.

7. The cell-free composition of claim 1, wherein the stimulating agent is one or a combination of a growth factor, an angiogenesis modulating agent, a hormone, a drug, a cell surface receptor, an enzyme, a nucleic acid.

8. The cell-free composition of claim 7, wherein the growth factor is one or a combination of: vascular endothelial growth factor (VEGF), a fibroblast growth factor (FGF), epidermal growth factor (EGF), platelet derived growth factor (PDGF), Transforming growth factors-beta (TGF-beta), transforming growth factor-alpha (TGF-alpha), an insulin-like growth factor, erythropoietin (Epo), tumor necrosis factor-alpha (TNF-alpha), tumor necrosis factor-beta (TNF-beta), interferon-gamma (INF-gamma), or a colony stimulating factor (CSFS).

9. The cell-free composition of claim 7, wherein the angiogenesis modulating agent is one or more of: angiopoietin-1, epidermal growth factor (EGF), hepatocyte growth factor (HGF), tumor necrosis factor (TNF-alpha), platelet derived endothelial cell growth factor (PD-ECGF), platelet derived growth factor (PDGF), insulin-like growth factor (IGF), interleukin-8 (IL-8), growth hormone (GH), angiopoietin, vascular endothelial growth factor (VEGF), a fibroblast growth factor (FGF), transforming growth factor alpha (TGF-alpha), CYR 61, or platelet-derived growth factor (PDGF).

10. The cell-free composition of claim 1, wherein the stimulating agent promotes production of a growth factor in the cell, modifies DNA of cells in the target tissue or causes gene expression of cells in the target tissue to be modified, promotes or enhances cell-cell communication, increases vessel density in the target tissue, promotes electrical signal transduction, causes activation of tissue remodeling, promotes cell proliferation, promotes electrical signal transduction, promotes cell differentiation, promotes cell survival, promotes energy production, activates cell repair pathways, or promotes secretion of secretory factors.

11.-22. (canceled)

23. The cell-free composition of claim 1, wherein the carrier allows for time-release of one or more therapeutic components.

24. The cell-free composition of claim 1, wherein the carrier is a film, a patch, a sponge, a foam, or a gel.

25.-29. (canceled)

30. The cell-free composition of claim 1, wherein the carrier comprises a biological matrix, a synthetic matrix, or a combination thereof.

31. The cell-free composition of claim 1, wherein the carrier comprises a collagen matrix, gelatin, or a combination thereof.

32.-35. (canceled)

36. The cell-free composition of claim 1, wherein the carrier helps contain the cell-free composition in the target tissue.

37. The cell-free composition of claim 1, wherein the target tissue is muscle, bone, cartilage, central nervous system tissue, eye tissue, epithelial tissue, gastrointestinal tissue, auditory tissue, genital tissue, oral cavity tissue, or connective tissue.

38.-39. (canceled)

40. The cell-free composition of claim 1, wherein the composition comprises two or more stimulating agents.

41.-56. (canceled)
